(19) United States
(12) Patent Application Publication
(10) Pub. No.: US 2025/0302541 A1
(43) Pub. Date: Oct. 2, 2025

(54) METHODS AND SYSTEMS FOR SURGICAL NAVIGATION AND INTRA-OPERATIVE SURGICAL PLANNING IN JOINT ARTHROPLASTY PROCEDURES USING PLANAR AND NON-PLANAR PROFILES

(71) Applicant: INTELLIJOINT SURGICAL INC., KITCHENER (CA)

(72) Inventors: RILEY AARON BLOOMFIELD, NEW HAMBURG (CA); JOSEPH ARTHUR SCHIPPER, KITCHENER (CA); ANDRE NOVOMIR HLADIO, WATERLOO (CA)

(21) Appl. No.: 18/617,347

(22) Filed: Mar. 26, 2024

(51) Int. Cl. A61B 34/20; A61B 34/00; A61B 34/10

(52) U.S. Cl. CPC A61B 34/20; A61B 34/10; A61B 34/25; A61B 2034/105; A61B 2034/107; A61B 2034/2055; A61B 2034/2068; A61B 2034/256

(57) ABSTRACT

Methods and systems are disclosed for improved surgical navigation and intra-operative surgical planning for joint arthroplasty procedures. A computing device receives tracking information of a patient's anatomic structure and of one or more surgical tools. A computing device further receives at least one plurality of anatomic points. A mesh is generated for each region of interest of the patient's anatomic structure from the one or more pluralities of anatomic points. One or more planar profiles and/or one or more non-planar profiles may be generated from each mesh and may be displayed to a user via a user interface. Planar and non-planar profiles may be updated as the user repositions a trackable cut plane on or adjacent the patient's anatomic structure, such as during resection planning during a TKA, for example.

### FIELD OF THE INVENTION

The present application relates to computer assisted surgical procedures and more particularly to methods and systems for surgical navigation and intra-operative surgical planning in joint arthroplasty procedures using planar and non-planar profiles.

### BACKGROUND

In Total Joint Arthroplasty (TJA), such as Total Hip Arthroplasty (THA) or Total Knee Arthroplasty (TKA), a surgeon may use a navigation system to assist in the surgery. An optical sensor (e.g. an image sensor) collects images of optically detectable trackers which are rigidly coupled to surgical instruments, implant components and the patient's anatomy. Navigation systems require the relevant portions of the patient's anatomy to be registered to define the spatial relationship between important features or landmarks of the patient's anatomy in real space and the navigation system's virtual coordinate system. Existing registration methods may involve rigidly attaching a first tracker directly to the patient's anatomy (e.g. to a tibia or a femur) and then requiring a surgeon to physically locate and touch several anatomic points on a patient anatomy with the tip of a probe attached to a second tracker to provide the position of anatomic data points to the navigation system.

Once registration is complete, the navigation system continuously estimates the position and location of the trackers and the objects to which they are attached by determining the pose of the trackers from the images. Using the relative locations and positions of trackers attached to a patient's bone and to one or more surgical tools, a navigation system can provide accurate measurements to assist the surgeon with the joint replacement procedure. For example, the measurements can provide guidance for intra-operative surgical planning for bone resections, gap balancing and implant placement.

It is desired to provide improved methods and systems for surgical navigation and intra-operative surgical planning of bone resections.

### SUMMARY OF THE INVENTION

Methods and systems are disclosed for improved surgical navigation and intra-operative surgical planning for joint arthroplasty procedures. A computing device receives tracking information of a patient's anatomic structure and of one or more surgical tools. A computing device further receives at least one plurality of anatomic points. A mesh is generated for each region of interest of the patient's anatomic structure from the one or more pluralities of anatomic points. One or more planar profiles and/or one or more non-planar profiles may be generated from each mesh and may be displayed to a user via a user interface. Planar and non-planar profiles may be updated as the user repositions a trackable cut plane (e.g. indicated by one or more trackable instruments) on or adjacent the patient's anatomic structure, such as during resection planning during a TKA, for example.

There is provided a computer-implemented method comprising: receiving tracking information of an anatomic structure of a patient; receiving tracking information of one or more surgical instruments; receiving at least one plurality of anatomic points identifying actual locations in one or more regions of the anatomic structure of the patient; generating a respective mesh of each of the one or more regions from the at least one plurality of anatomic points; generating at least two planar profiles from one or more respective meshes, wherein each planar profile is defined to be a slice that is parallel to one or both of an anatomic axis or an anatomic reference plane; displaying the at least two planar profiles simultaneously via a user interface; receiving updated tracking information of the anatomic structure and the one or more surgical instruments; updating the at least two planar profiles according to the updated tracking information; and displaying the at least two updated planar profiles on a user interface.

The method may comprise determining a dynamic distal point of each respective mesh relative to a trackable cut plane indicated by the one or more surgical instruments based on the tracking information of the anatomic structure and the tracking information of the one or more surgical instruments; and each planar profile from one respective mesh may comprise the dynamic distal point of the one respective mesh.

Receiving the at least one plurality of anatomic points may comprise determining a location of a probe tip of the one or more surgical instruments for each of the points of the at least one plurality of anatomic points. Receiving the at least one plurality of anatomic points may comprise receiving input from a scanner being manipulated to scan a region of the patient anatomy.

The one or more surgical tools may comprise at least two surgical tools; wherein a first surgical tool comprises the probe tip and a second surgical tool indicates the trackable cut plane. Alternatively, the one or more surgical tools may comprise one surgical tool; wherein the one surgical tool comprises the probe tip and indicates the trackable cut plane.

The trackable cut plane may be indicated by a probe base and/or one or more of a cutting guide and a paddle guide.

The anatomic structure may be a tibia and the one or more regions may comprise at least one of a medial tibial plateau and a lateral tibial plateau. One or more planar profiles may be defined by a slice that is parallel to both a mechanical axis of the tibia and an anterior-posterior (AP) axis of the tibia. Additionally or alternatively, one or more planar profiles may be defined by a slice that is parallel to both a mechanical axis of the tibia and a medial-lateral (ML) axis of the tibia.

The anatomic structure may be a femur and the one or more regions may comprise at least one of a medial femoral condyle, a lateral femoral condyle, and a posterior condyle. One or more planar profiles may be defined by a slice that is parallel to both a mechanical axis of the femur and Whiteside's line. Alternatively or additionally, one or more planar profiles may be defined by a slice that is parallel to both a mechanical axis of the femur and an ML axis of the femur.

The method may further comprise determining a resection depth for each of the one or more regions based on one or more planar profiles and displaying at least one resection depth via a user interface.

One or more meshes may comprise interpolated points.

The method may further comprise displaying a first heat map for a first respective mesh via a user interface; wherein the first heat map displays a position of either or both of the anatomic points and the interpolated points of the first respective mesh relative to either a) the trackable cut plane or b) a mechanical axis of the anatomic structure

According to another broad aspect, there is provided a computer-implemented method comprising: receiving tracking information of an anatomic structure of a patient; receiving tracking information of one or more surgical instruments; receiving at least one plurality of anatomic points identifying actual locations in one or more regions of the anatomic structure of the patient; generating a respective mesh for each of the one or more regions from the at least one plurality of anatomic points; generating a non-planar profile for each respective mesh; and displaying one or more non-planar profiles via the user interface.

According to an aspect, generating at least two planar profiles from each respective mesh; wherein each planar profile is defined by a slice that is parallel to either or both of: an anatomic axis or an anatomic reference plane; determining either: 1) respective dynamic distal points of each of the at least two planar profiles relative to a trackable cut plane indicated by the one or more surgical instruments based on the tracking information of the anatomic structure and the tracking information of the one or more surgical instruments; or 2) respective anatomic distal points of each of the at least two planar profiles; and assembling the non-planar profile for each respective mesh comprising either: 1) the respective dynamic distal points; or 2) the respective anatomic distal points.

According to another aspect, generating the one or more non-planar profiles may comprise: dividing each mesh into at least two areas; determining respective anatomic distal points of each area; or respective dynamic distal points of each area for the non-planar profile for the one respective mesh.

Any computer-implemented method disclosed herein may have a corresponding computer system. For example, a computer system may comprise at least one processing unit and a memory coupled to at least one processing unit, a storage device storing instructions that, when executed by the at least one processing unit, cause the computer system to perform operations of any computer-implemented method described herein.

### BRIEF DESCRIPTION OF THE DRAWINGS

FIG. 1 is an overview of navigated TKA, in accordance with an embodiment, for example, where a computing device, such as a laptop, is configured as disclosed herein.

FIGS. 2A-2D illustrate various configurations of trackable probes from prior art. FIG. 2A illustrates a prior art trackable probe with both a probe tip and a probe base. FIG. 2B illustrates prior art paddles and cutting guide. FIG. 2C illustrates a prior art trackable probe coupled to a cutting guide, in accordance with an embodiment. FIG. 2D illustrates a prior art trackable probe coupled to a cutting guide and paddles in accordance with an embodiment.

FIG. 3A illustrates a proximal tibia with anatomic points overlaid. FIG. 3B illustrates a distal femur with anatomic points overlaid.

FIG. 4 is an exemplary graphical user interface (UI) that may be used for registration of a proximal surface of a tibia of a patient in accordance with an embodiment.

FIG. 5A is an exemplary graphical UI that may be used for tibial resection planning, displaying heat maps and planar profiles, in accordance with an embodiment.

FIG. 5B is an exemplary graphical UI that may be used for tibial resection planning, displaying heat maps and planar profiles for an example where lift-off has occurred during data collection for registration of the proximal surface of the tibia, in accordance with an embodiment.

FIG. 6A is an exemplary graphical UI that may be used for tibial resection planning, displaying planar profiles overlaid on generic representations of a proximal tibia and a distal femur, according to an embodiment.

FIG. 6B is an exemplary graphical UI that may be used for femoral resection planning, displaying planar profiles overlaid on generic representations of a proximal tibia and a distal femur, according to an embodiment.

FIG. 7A illustrates a mesh comprising anatomic and/or interpolated points, in accordance with an embodiment wherein the mesh is divided into multiple areas. FIG. 7B illustrates anatomic distal points determined for each area of a mesh, in accordance with an embodiment. FIG. 7C illustrates a non-planar profile generated from a collection of distal points, in accordance with an embodiment.

FIG. 8 illustrates a mesh comprising anatomic and/or interpolated points and multiple non-planar profiles, in accordance with an embodiment.

FIG. 9 is a flow chart of operations of a computing device illustrating respective computer-implemented methods in accordance with embodiments disclosed herein.

FIG. 10 is a flowchart of operations of a method for generating a non-planar profile for at least one mesh, in accordance with an embodiment.

FIG. 11 is a flowchart of operations of a method for generating a non-planar profile for at least one mesh, in accordance with an embodiment.

It will be appreciated that for simplicity and clarity of illustration, elements shown in the figured have not necessarily been drawn to scale. For example, the dimensions of some of the elements may be exaggerated relative to other elements for clarity.

### DETAILED DESCRIPTION

Described herein are systems and methods for performing a navigated surgical procedure involving a patient's anatomy. The primary example disclosed herein is a navigation-assisted TKA. However, it should be evident that the systems, devices, apparatuses, methods and computer-implemented methods described herein may be applied to any anatomy requiring treatment (e.g. a cranium, a spine, a pelvis, a femur, a tibia, a hip, a shoulder, or an ankle).

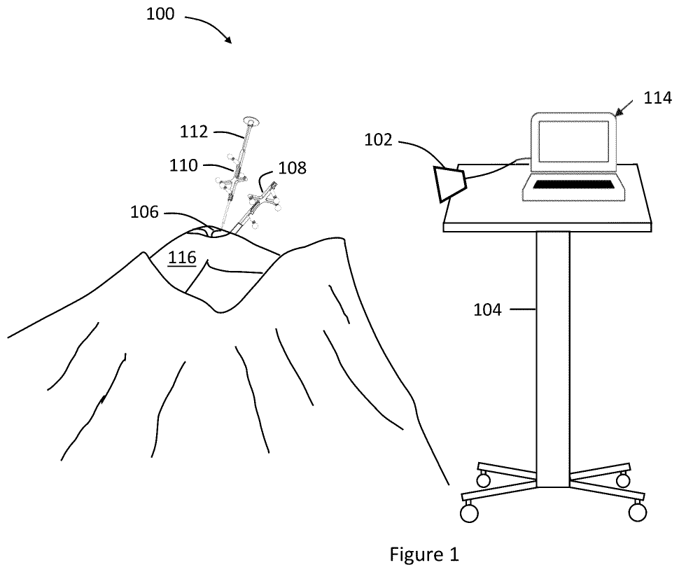

FIG. 1 illustrates an exemplary intra-operative navigation system 100, in the context of a navigated TKA. In this intra-operative navigation system 100, an image sensor 102 is shown located on a moveable cart 104, with its field of view oriented towards a surgical site 106. The image sensor 102 could alternatively be mounted on an anatomic structure of the patient, held in the hands of the operator, coupled to a mounting arm or structure or any other appropriate position. Image sensor 102 comprises one or more sensor devices, for example, a camera for determining image sensor data. Other sensors of device 102 may comprise devices for determining directional information such an accelerometer, gyroscope, etc.

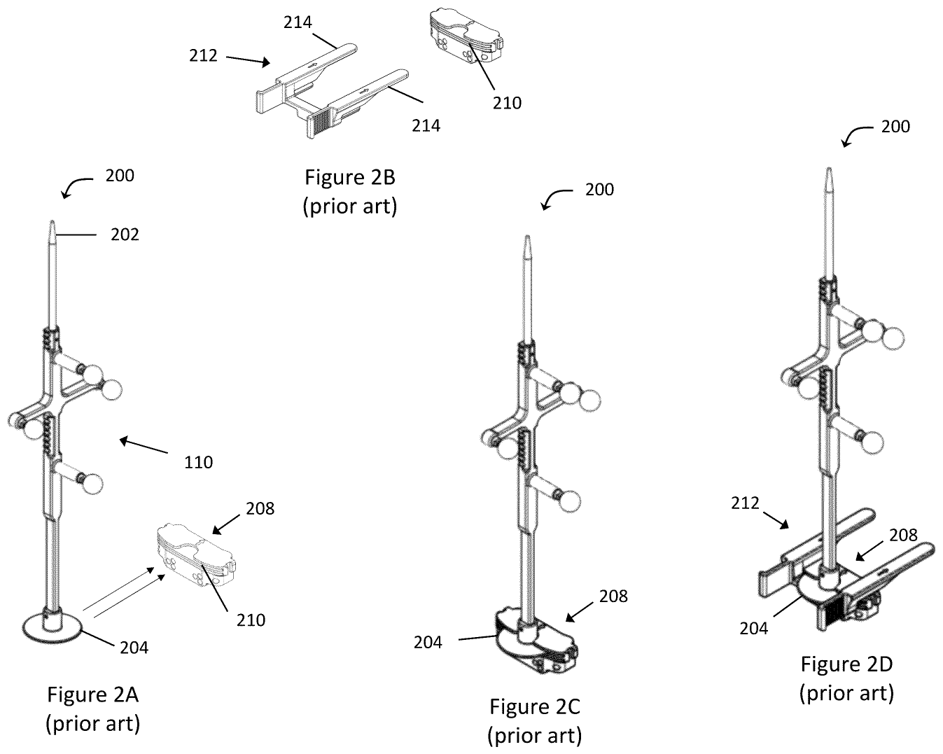

One or more trackers may be attached to various objects, including an anatomic structure (bone) of a patient and/or a surgical instrument; the one or more trackers providing optically detectable features for detection by the image sensor 102. In the embodiment shown in FIG. 1, a first tracker 108 is coupled to an anatomic structure of a patient (i.e. a tibia or a femur) and a second tracker 110 is coupled to a surgical instrument 112, which in this case is a probe. A surgical instrument 112 may be any type of instrument used in a surgical environment, such as a probe, a tool for cutting or resecting tissue, or a cutting guide 208 (see FIG. 2). The skilled person will understand that there can be any number of trackers coupled to any number of anatomic structures and/or surgical instruments.

Where the navigation system 100 comprises two or more trackers, the trackers may be identical to each other. In an embodiment, the two or more trackers may have different optically detectable features such that the navigation system can differentiate the trackers. For instance, the trackers may have different colors, geometries, sizes, or numbers and/or arrangement of optically detectable features (e.g. retro-reflective spheres as shown in FIGS. 2A-D).

Image sensor 102 transmits image sensor data (including image data or pose data associated with the trackers, such as tracker 108 and/or 110) to a computing device 114. Image sensor 102 may be communicatively coupled to computing device 114 by wire (as shown). Alternatively, communication between image sensor 102 and computing device 114 may be wireless communication. The computing device 114 may comprise a laptop, workstation, or other computing device having at least one processing unit and at least one storage device such as memory storing software (instructions and/or data) as further described herein to configure the execution of the computing device such as to perform operations of a method. System 100 may comprise one or more computing devices. A computing device may comprise a cloud server and/or remote computing devices.

Computing device 114 performs applicable processing to calculate the poses of one or more trackers. Where the trackers have a known spatial or geometrical relationship to a coupled object, such as via registration, computing device 114 also performs the applicable processing to calculate the pose of the coupled objects. For example, where a tracker, such as tracker 108, is coupled and registered to the anatomic structure of a patient 116, the pose of the anatomic structure of the patient may be determined by computing device 114. Further, where a tracker, such as tracker 110, is coupled to a surgical instrument, such as surgical instrument 112, computing device 114 may determine the pose of the surgical instrument using the known spatial relationship between the tracker and the surgical instrument. Computing device 114 may further determine a relative pose between two or more objects, such as between a surgical instrument and an anatomic structure of a patient's anatomy 116 (e.g. femur or tibia). Pose may be determined in three dimensions and comprises position, location and/or orientation of an object. The computing device 114 may further display clinically relevant information to the user, including tracking information, wherein tracking information may comprise image data and/or pose data associated with the pose of one or more trackers and one or more objects to which the trackers are coupled. For example, tracking information may comprise image data and/or pose data associated with trackers 108 and 110 and the objects to which they are coupled, such as surgical instrument 112 and an anatomic structure of a patient's anatomy 116, respectively.

Referencing FIGS. 2A-2D, the surgical tool may be a trackable probe 200, comprising a tracker, such as tracker 110, a probe tip 202 at one end and a probe base 204 at another end. In an embodiment, the probe base 204 may be couplable to a cutting guide 208 (see FIGS. 2A and 2C) and/or a paddle guide 212 (see FIG. 2D) See the probe base and cutting guide as disclosed in Applicant's U.S. patent application Ser. No. 17/291,526, published as US20220000564 A1 on Jan. 6, 2022, and entitled, “Methods and Systems for Surgical Navigation and Devices for Surgery”, which is incorporated herein by reference in its entirety. In an embodiment, probe base 204 may comprise a planar disc oriented such that the disc lies perpendicular to the central axis of trackable probe 200. Probe base 204 may be coupled to cutting guide 208 as shown in FIGS. 2C and 2D. The thickness of probe base 204 may correspond to the thickness of a slot 210 of cutting guide 208 such that probe base 204 may be inserted into a slot 210 of cutting guide 208. When coupled to cutting guide 208 in this manner, the position of probe base 204 may represent (e.g. indicate) the cut plane for the purpose of positioning cutting guide 208 while planning a resection, as further discussed below. Movement of the probe may be tracked and the updated position of the probe's base may indicate an updated cut plane to provide a tracked cut plane). The shape of probe base 204 as disclosed herein is round, however probe base 204 may be any shape, such as triangular, square, rectangular, elliptical, or any other suitable shape for coupling with cutting guide 208. Probe base 204 may be coupled to cutting guide 208 magnetically. Further, paddle guide 212 may be coupled to cutting guide 208 to facilitate positioning the cutting guide on or adjacent to the anatomic structure of the anatomic structure of the patient to be resected. Cutting guide 208 and paddle guide 212 may be coupled magnetically in accordance with an embodiment via the coupling of two or more paddles 214 with a slot 210 of cutting guide 208. The skilled person will appreciate that the invention is not limited to the use of cutting guide 208 and/or paddle guide 212 disclosed herein, but that any cutting guide and/or paddle guide may be used, provided the probe base, the cutting guide and the paddle guide are configured appropriately for coupling together. Further, the skilled person will also appreciate that a cutting guide may be coupled directly to a tracker or may be formed integrally with a tracker (i.e. a trackable cutting guide). An integrated tracker and cutting guide may be coupled or integrally combined with a paddle guide.

In an embodiment, there may be two or more trackable surgical instruments. In an embodiment, a first trackable surgical instrument is a trackable probe comprising probe tip 202 and a second trackable surgical instrument is a trackable probe comprising probe base 204. In another embodiment, a first trackable surgical instrument is a trackable probe comprising probe tip 202 and a second trackable surgical instrument is a trackable cutting guide. The skilled person will understand that there may be any number of trackable surgical instruments with any combination of features.

In order to determine the pose of probe tip 202 and/or probe base 204, the geometry of tracker 110 relative to probe tip 202 and probe base 204 should be known to the system. This can be achieved by designing and manufacturing probe tip 202, tracker 110 and probe base 204 as separate components that can only be assembled in a single unique configuration, such as trackable probe 200. In an embodiment, trackable probe 200 comprising probe tip 202, tracker 110, and probe base 204, may be designed and manufactured as a single integral component. In another embodiment, registration or calibration steps can be performed to determine the spatial relationships between probe tip 202 and tracker 110 and between probe base 204 and tracker 110.

Intra-operative navigation system 100 is registered to patient's anatomy 116; that is, the positional and geometric relationships between the patient's anatomic planes/axes/features/landmarks are known to computing device 114. It is understood that the camera and objects are registered to surgical navigation system 100 in accordance with a registration procedure or procedures. For example, a method and system for surgical navigation has been disclosed in Applicant's U.S. Patent U.S. Pat. No. 9,247,998 B2, granted Feb. 2, 2016, and entitled “System and Method of Intra-Operative Leg Position Measurement”, the content of which is incorporated herein by reference in its entirety.

In the embodiment shown in FIG. 1, computing device 114 is shown sitting on a movable cart 104 and comprising a keyboard and a display, which input and output devices are coupled to the computing device. Not shown for the computing device are at least one processing unit and a storage device such as memory that stores instructions that, when executed by the at least one processing unit, cause computing device 114 to preform various functions and operations, for example, in accordance with the method aspects shown and described.

The various computing devices included herein can comprise one or more processing units (for example a microprocessor, FPGA, ASIC, logic controller, or any other appropriate processing hardware), a storage device (e.g. non-transitory processor-readable storage medium, such as memory, RAM, ROM, magnetic-disk, solid state storage, or any other appropriate storage hardware) storing instructions that, when executed by the processing unit, cause the computing device to perform operations of a computer-implemented method, for example, to provide the functionality and features described herein. Computer program code for carrying out operations may be written in any combination of one or more programming languages, e.g., an object-oriented programming language such as Java, Smalltalk, C++ or the like, or a conventional procedural programming language, such as the “C” programming language or similar programming languages.

Any of the computing devices may have communication subsystems to communicate via a network. Any may have a display device and other input and/or output devices.

Though the description herein is generally set out in relation to a TKA, it will be understood to a person of ordinary skill in the art that the teachings herein can be applied to other joints, such as the bones of a hip, a shoulder joint, or an elbow joint.

In a navigation-assisted TKA, both the tibia and femur are registered to navigation system 100. Either the tibia or the femur can be registered first according to either a tibia first or femur first surgical workflow, both of which are known in the art.

To register one or more anatomic structures of the patient, a user, typically a surgeon or another member of the surgical team, touches probe tip 202 of a trackable probe, such as trackable probe 200 to a series of actual locations on the anatomic structure of the patient while image sensor 102 transmits image data associated with trackable probe 200, and a tracker coupled to the patient's anatomic structure, such as tracker 108. In FIG. 1, image sensor 102 transmits the image data to computing device 114. Computing device 114 performs the necessary calculations to determine the location of probe tip 202 based on the pose of trackable probe 200 relative to the pose of the anatomic structure of the patient (based on the pose of tracker 108). The location of probe tip 202 therefore identifies the location of actual anatomic points on the anatomic structure of the patient relative to tracker 108 coupled to the patient.

Registration may involve touching probe tip 202 to a single discrete actual location or a series of single discrete actual locations, such as when an anatomic landmark or reference location can be identified by a single point or a series of single points. For example, to register the mechanical axis of the tibia, the user may touch probe tip 202 to each of three actual locations of the anatomic structure, namely the lateral and medial malleoli of the ankle and the tibia center located on the proximal surface of the tibia. Similarly, the anterior-posterior (AP) axis of the patient's tibia may be defined by touching probe tip 202 to the posterior cruciate ligament (PCL) insertion point and the one-third medial tubercle.

Alternatively, or in addition, registration may comprise tracing probe tip 202 along a surface of an anatomic structure, such that a plurality of anatomic points is used to register an anatomic landmark or reference location. For example, an anatomic landmark or reference location may be a surface or a portion of a surface (i.e. a region) comprising an anatomic landmark. An anatomic structure may comprise one or more regions.

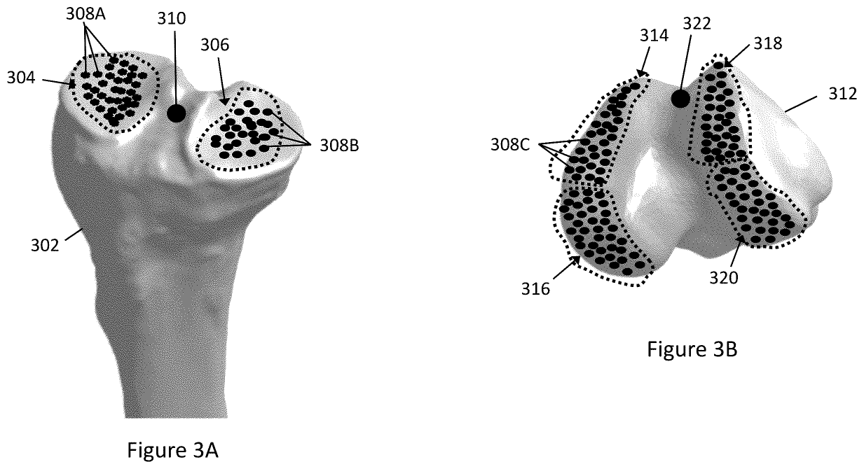

FIG. 3A illustrates a perspective view of a tibia of a patient, in accordance with an embodiment involving registration of a tibia. As shown in FIG. 3A, a proximal tibia 302 may comprise the medial tibial plateau 304 (i.e. a first region) and the lateral tibial plateau 306 (i.e. a second region). In FIG. 3A, the first and second regions are indicated approximately by dashed lines. According to an embodiment, the tibial plateaus may be registered by tracing probe tip 202 along the surface of each tibial plateau in a scribbling, tracing or painting motion. During tracing, the image sensor 102 transmits tracking information (image data and/or pose data associated with the pose of trackable probe 200 and tracker 108 coupled to the anatomic structure of the patient) to computing device 114 as probe tip 202 is traced along the surface of the anatomic structure. Tracking information may be transmitted continuously to computing device 114 as probe tip 202 is traced along the surface of an anatomic structure. Computing device 114 performs the necessary processing to calculate the pose of trackable probe 200 and tracker 108 to determine the location of each of a plurality of anatomic points, such as anatomic points 308A/B/C, by determining the location of probe tip 202 as it is traced along the bone surface. Thus, each of the anatomic points identifies an actual location of the anatomic structure of the patient.

A plurality of points may identify actual locations in one or more regions of the anatomic structure. Referring to FIG. 3A, in accordance with an embodiment involving registration of a tibia, one or more pluralities of anatomic points may identify actual locations in one or more regions of a patient's tibia. In the embodiment shown, a first plurality of anatomic points 308A may identify actual locations in a first region, such as the medial tibial plateau 304. In an embodiment, a second plurality of anatomic points 308B may identify actual locations in a second region, such as the lateral tibial plateau 306. In another embodiment, a first plurality of anatomic points may identify actual locations in both a first region and a second region of a tibia of the patient, such as the medial and lateral tibial plateaus 304 and 306. The skilled person will appreciate that additional regions of the tibia, other than the medial and lateral tibial plateaus may also be registered via tracing or painting.

FIG. 3B illustrates a perspective view of a femur of a patient, in accordance with an embodiment involving registration of a femur. In an embodiment, one or more pluralities of anatomic points may identify actual locations in one or more regions of a patient's femur 312. In the embodiment shown, a plurality of anatomic points 308C may identify actual locations in multiple regions. In an embodiment, a plurality of anatomic points may identify actual locations in one or more of a first, second, third, and fourth regions, such as a distal portion of the medial femoral condyle 314, a posterior portion of a medial femoral condyle 316, a distal portion of a lateral femoral condyle 318 and a posterior portion of a lateral femoral condyle 320, respectively. In another embodiment, a first plurality of anatomic points may identify actual locations in a first region (i.e. a distal portion of a medial femoral condyle 314), a second plurality of anatomic points may identify actual locations in a second region (i.e. a posterior portion of a medial femoral condyle 316), a third plurality of points may identify actual locations in a third region (i.e. a distal portion of a lateral femoral condyle 318) and a fourth plurality of points may identify actual locations in a fourth region (i.e. a posterior portion of a lateral femoral condyle 320). The skilled person will appreciate that additional regions of the femur, other than the medial and lateral tibial plateaus may also be registered via tracing or painting.

The skilled person will readily appreciate that a plurality of anatomic points may identify actual locations in any number of regions and/or that there may be more than one plurality of anatomical points, wherein each plurality of anatomic points identifies actual locations in one or more regions. In this way, one or more surfaces or regions of an anatomic structure may be registered instead of or in addition to registration of individual anatomic landmarks or reference locations. The skilled person will also appreciate that a plurality of anatomic points may identify actual locations in regions of interest other than those specifically disclosed herein, such as areas of lowered or raised topography of the surface of the anatomic structure. An example of lowered topography could be cartilage wear.

The location of an anatomic point may be captured via a user's interaction with navigation system 100, such as when a user presses a button on a mouse, presses a key on a keyboard, provides a voice command, presses a foot pedal, or touches a portion of a touchable screen, etc., to capture the anatomic point while the user holds probe tip 202 in a desired position or location (pose).

In an embodiment, capturing the locations of each of a plurality of points may be triggered to begin manually by pressing a key on a keyboard, clicking a button on a mouse, clicking a button on a remote control, using audio commands, pressing a foot pedal, or pressing a portion of a touch screen. In an embodiment, data collection may continue until a sufficient number of points have been collected. For instance, 40 points, 80 points, or any other appropriate number of points may be collected before data collection automatically ends. The number of points collected may be pre-defined in the navigation system, or may be user configurable, such as via a drop-down menu or manual user entry via typing on a keyboard. Alternatively, data collection may be triggered to end based on a set of data quality criteria, such as surface smoothness, point density within a given area (i.e. a number of points captured within a given surface area, etc.). In an embodiment, data collection may be triggered to end manually by pressing a key on a keyboard, clicking a button on a mouse, clicking a button on a remote control, using audio commands, pressing a foot pedal, or pressing a portion of a touch screen.

In an embodiment, one or more pluralities of anatomic points may be received from a 3D surface scanning system and registered to navigation system 100, as described in the Applicant's U.S. Patent U.S. Pat. No. 11,432,878 B2, entitled “Systems, Methods and Devices to Scan 3D Surfaces for Intra-operative Localization”, issued Sep. 6, 2022, the content of which is incorporated herein by reference in its entirety. The 3D surface scanning system may use a laser or a camera to acquire one or more pluralities of points. Where the 3D surface scanning system uses a camera, camera 102 of navigation system 100 may be used for both navigation and surface scanning.

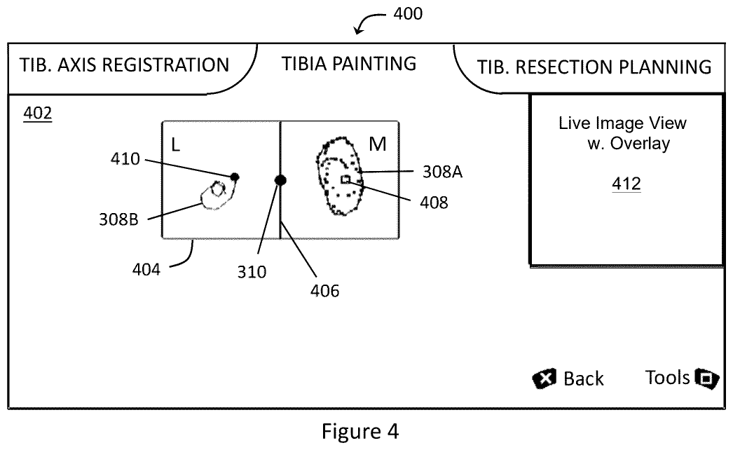

As shown in FIG. 4, in accordance with an embodiment, computing device 114 may display the plurality of anatomic points 308A/B or a subset of a plurality of points to the user via a user interface (UI) 400. FIG. 4 displays anatomic points 308 A/B representing actual locations of the proximal end of the tibia in plan view (i.e. perpendicular to the mechanical axis of the tibia). Computing device 114 may continuously receive a plurality of anatomic points in real-time as probe tip 202 traces the surface of the anatomic structure. Computing device 114 may display the plurality of anatomic points 308A/B to the surgeon or other user, for example, via tab 402 of UI 400. In the embodiment shown, a first plurality of anatomic points 308A identifying actual locations in a first region (i.e. the medial tibial plateau 304) and a second plurality of anatomic points 308B identifying actual locations in a second region (i.e. the lateral tibial plateau 306) are displayed. As previously discussed, there may alternatively be a single plurality of points comprising the anatomic points of the first region and the second region. The skilled person will appreciate that any appropriate number of pluralities of anatomic points identifying actual locations in any appropriate number of regions may be displayed via UI 400. Further, the skilled person will understand that a UI, such as UI 400, could alternatively, or in addition, display one or more pluralities of points identifying actual locations in one or more regions of a patient's femur, such as the regions previously discussed in relation to FIG. 3B. In the embodiment, UI 400 displays a live image view with an overlay 412. The live image view comprises (or is derived from) images from optical sensor 102, for example, showing the patient anatomy (e.g. a surgical site 106 of a tibia), tracker 108 therefor and the surgical tool 112 (e.g. including its tracker 110) probing the tibia to paint it. In an embodiment, images for display may be derived by processing data from the image sensor 102 prior to their display. Rendered to the “live” images for display is the overlay. In an embodiment, the overlay annotates patient anatomy (e.g. the tibia) providing a line between two points associated with the tibia. In an embodiment, the line represents an (e.g. mechanical) axis, such as the AP axis. In an embodiment, the overlay further includes a text label.

In FIG. 4, anatomic points 308A/B (identifying actual locations in the lateral and medial tibial plateaus) are displayed side by side (i.e. the lateral tibial plateau on the left and the medial tibial plateau on the right). The skilled person will appreciate that other display arrangements are possible, wherein anatomic points identifying locations in a first and second region may be displayed such that the first region is positioned above the second region or vice versa. Further, in FIG. 4, the anatomic points 308B/A representing actual locations in the lateral and medial tibial plateaus, respectively, are displayed to correspond to their relative locations on the actual patient anatomy from the viewing perspective of the user, such as a surgeon. In the illustrated embodiment, the patient's right knee is being operated on. The skilled person will understand that where a TKA is being performed on a patient's left knee, the medial tibial plateau would be located on the left and the lateral tibial plateau would be located on the right. Further, in an embodiment where the femur is being operated on, more than two regions may be displayed to the surgeon, such as the regions previously discussed in relation to FIG. 3B. In an embodiment, UI 400 may be programmed to automatically display one or more regions in alignment with the viewing perspective of the user. In an embodiment, the user selects the left or right knee in the UI at the start of the procedure by clicking a mouse button, touching the appropriate portion of a touchscreen, pressing a key on a keyboard, via voice commands, etc. In an embodiment, the one or more regions are not displayed according to the viewing perspective of the user. In an embodiment, the configuration of the display may be customized by the user in a preferred configuration using a drop-down menu, using a mouse to perform a drag-and-drop operation, or by providing user inputs via a keyboard.

In FIG. 4, the first plurality of points 308A in the first region is shown following completion of data collection for the first region. The second plurality of points 308B in the second region is shown at an instant in time when the user is tracing probe tip 202 along the lateral tibial plateau 306 of the patient, but the second plurality of points has not yet been completely collected. The first and/or second pluralities of points may be displayed as a series of points represented by dots (e.g. closed circles) or any other shape. In an embodiment, a plurality of points may be displayed as a continuous line connecting at least some of the points (the points may or may not be visible). In an embodiment, a plurality of anatomic points may be displayed as a series of points with an overlay of a continuous line connecting at least some of the points, as shown for the first plurality of anatomic points 308A. Probe tip's current location 410 (i.e. the location of the probe tip 202 at the instant shown in FIG. 4) may be shown by a graphical indicator, such as a closed circle or dot. The skilled person will understand that the graphical indicator could be any shape, such as an open circle, a dot or closed circle (as shown), a square, a triangle, a star, an x, a plus sign, or any other appropriate shape.

In an embodiment, one or more pluralities of points may be displayed relative to anatomic landmarks or other reference points of the anatomic structure. For example, in the embodiment of FIG. 4, the first and second pluralities of points 308A and 308B, respectively, are shown relative to an anatomic point identifying tibia center 310 (see also FIG. 3A for approximate location of tibia center), indicated by a graphical indicator in the shape of a closed circle. The skilled person will understand that the graphical indicator could be any appropriate shape, as discussed previously. The one or more pluralities of anatomic points may also be shown relative to any other anatomic landmark, such as AP axis 406 of the tibia, depicted as a single solid line in FIG. 4. The skilled person will understand that the graphical indicator could be any shape, such as a dashed line, a double line, a series of closed or open circles, triangles, stars, plus signs, or any other appropriate shape. Where the anatomic structure is a femur, other anatomic landmarks may be displayed, such as the femur center 322 (see FIG. 3B for approximate location) and/or Whiteside's line.

In an embodiment, a plurality of anatomic points or a subset of a plurality of anatomic points may be assembled into a mesh of connected points to provide a discretized representation of an anatomic surface of the patient. The mesh can be used to locate or define anatomic landmarks and/or perform calculations for the registered anatomic structure, such as a resection depth. In an embodiment, computing device 114 generates a mesh from a plurality of anatomic points identifying actual locations in a region of the anatomic structure. In an embodiment, a separate mesh may be generated for each region of an anatomic structure.

In an embodiment, computing device 114 may generate a first mesh from a first plurality of points identifying actual locations in a first region and a second mesh from a second plurality of points identifying actual locations in a second region. The skilled person will readily appreciate that computing device 114 may generate any number of meshes, wherein each mesh is generated from a separate plurality of points identifying actual locations in a different region.

In another embodiment, computing device 114 may generate first and second meshes identifying actual locations in first and second regions, respectively, from a (single) first plurality of points. Computing device 114 may delineate the first and second meshes using anatomic information, such as anatomic landmarks, anatomic axes, and anatomic reference planes of the anatomic structure of the patient. In an embodiment, computing device 114 may define first and second subsets of points from the first plurality of points that are used to assemble the first and second meshes, respectively. Alternatively, computing device 114 may assemble a single mesh from a single plurality of points and then delineate two or more meshes corresponding to two or more regions, respectively. In the context of a patient's tibia, for example, computing device 114 may delineate a first mesh for the lateral tibial plateau 306 and a second mesh for the medial tibial plateau 304 from a first plurality of anatomic points. The skilled person will readily appreciate that computing unit 114 may generate a mesh for each of any number of regions from a single plurality of points. In the context of a patient's femur, for example, computing device 114 may delineate a separate mesh for each of: a distal portion of the medial femoral condyle 314, a posterior portion of a medial femoral condyle 316, a distal portion of a lateral femoral condyle 318 and a posterior portion of a lateral femoral condyle 320, respectively.

A mesh may comprise a plurality of anatomic points and the points may be evenly spaced or unevenly spaced. For example, points may be spaced more closely together in anatomic regions where the surface has more complex or more highly variable geometry and the points may be spaced further apart in anatomic regions where the surface is less complex or has simpler geometry. Further, computing device 114 may perform calculations to increase the spatial resolution of the mesh by interpolating between the anatomic points. A mesh may therefore further comprise a plurality of interpolated anatomic points.

In an embodiment involving the proximal tibia (i.e. such as when preparing to perform a proximal tibial cut), a first mesh may be generated for a medial tibial plateau 304 (i.e. a first region) and a second mesh may be generated for a lateral tibial plateau 306 (i.e. a second region). Alternatively or in addition, in an embodiment involving the distal femur (i.e. such as when preparing to perform a distal femoral cut), a first mesh may be generated for a distal portion of a medial femoral condyle 314 (i.e. a first region), a second mesh may be generated for a posterior portion of a medial femoral condyle 316 (i.e. a second region), a third mesh may be generated for a distal portion of a lateral femoral condyle 318 (i.e. a third region), and a fourth mesh may be generated for a posterior portion of a lateral femoral condyle 320 (i.e. a fourth region).

In an embodiment, computing device 114 may perform calculations using a mesh to locate one or more anatomic reference points and/or to calculate specific parameters used for intra-operative surgical planning, such as resection planning. Locating an anatomic reference point using a mesh may be more accurate and/or may provide workflow efficiency over locating an anatomic reference point via registration of a single anatomic point or a series of single anatomic points. Further, calculating specific parameters using a mesh, such as for resection planning, may be more accurate compared to calculating specific parameters using a single registered anatomic point.

In accordance with an embodiment, computing device 114 may perform calculations using the mesh to determine one or more anatomic distal points for each region. For example, a tibial anatomical distal point may be determined for each of the medial and lateral plateaus 304 and 306, respectively, by selecting a point from the mesh for each plateau that is most distal in a direction parallel to the mechanical axis of tibia 302, wherein the mesh comprises the respective plurality of points and may further comprise interpolated points. For example, an anatomic medial tibial distal point 408 may be determined via selection from the mesh for the first region. The resulting anatomic medial tibial distal point 408 may be displayed to the user, shown as a square-shaped graphical indicator in FIG. 4. The graphical indicator could be any other appropriate shape, as discussed previously regarding graphical indicators.

Alternatively or in addition, computing device 114 may perform calculations using respective meshes to determine one or more femoral anatomic distal points for regions corresponding to each of the distal portions of the medial and lateral femoral condyles 314 and 318, respectively. The femoral anatomic distal points may be determined for each of the medial and lateral femoral condyles by selecting a point from the respective mesh for each of the distal portions of the femoral condyles that is most distal in a direction parallel to the mechanical axis of the femur.

In an embodiment, computing device 114 may perform calculations using one or more meshes to determine one or more other anatomic points of interest. For example, computing device 114 may perform calculations to determine one or more femoral anatomic posterior points for regions corresponding to each of the posterior portions of the medial and lateral femoral condyles 316 and 320, respectively. The femoral anatomic posterior points may be determined for each of the medial and lateral femoral condyles by selecting a point from the respective mesh for each of the posterior portions of the femoral condyles that is most posterior. Traditionally, the anatomic distal and/or posterior points may be located by visual inspection and registration, wherein the user touches probe tip 202 to a single actual location. However, it can be challenging to accurately identify an anatomic distal and/or posterior point via visual inspection, particularly the anatomic distal points for the medial and lateral tibial plateaus due to their naturally gradual contouring. Additionally, osteophytes or wear patterns can obscure the anatomic distal and/or posterior points of the tibia and/or the femur. Uncertainty in locating these points can affect the surgeon's confidence in the navigation system, which in turn, can result in the surgeon repeating steps to increase their confidence, slowing down the procedure.

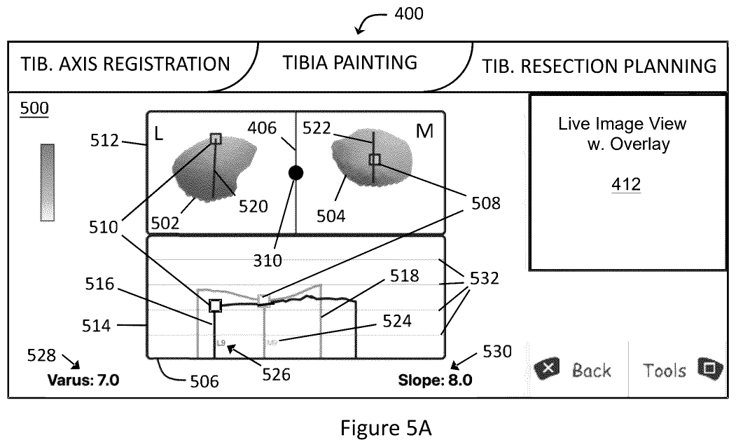

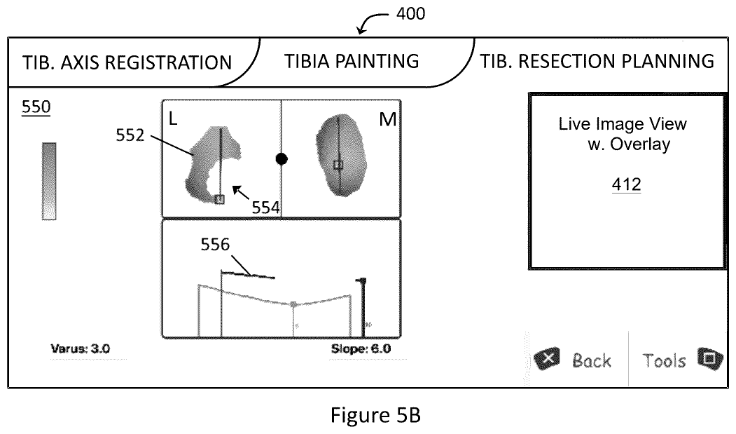

A mesh may be displayed to a user via UI 400, in accordance with an embodiment, for example, in the form of a heat map, as shown in FIG. 5A. UI 400 comprises tab 500, which includes displays to assist with resection planning. The upper portion of tab 500 may include heat maps, such as those included in display box 512. The lower portion of tab 500 may include additional displays, such as those included in display box 514, discussed in more detail below. The skilled person will understand that in some embodiments, only display box 514 is included, or alternatively that only display box 512 is included. Further, the skilled person will understand that display boxes 514 and 512 may be included on separate tabs of a UI. Further, there may be any number of display boxes containing any number of visualizations or information relevant to the procedure.

In the embodiment shown in FIG. 5A, computing device 114 may generate heat maps 504 and 502 for the meshes generated for the patient's medial and lateral tibial plateaus, respectively (i.e. a first mesh generated for the first region and a second mesh generated for the second region, discussed previously). Although not shown, computing device 114 may, alternatively or in addition, generate heat maps for the meshes generated for one or more regions of the patient's femur (i.e. regions of the femur previously discussed in relation to FIG. 3B). The heat map's color gradations represent the position of each point in the mesh along an axis parallel to the mechanical axis of the anatomic structure (i.e. in the distal-proximal direction). Alternatively, the heat map's color gradations may represent the position of each point relative to any other anatomic axis, anatomic reference plane, or other reference axis or reference plane. The anatomic axis, anatomic reference plane, or other reference axis or reference plane may be selected by the user from a static list or drop-down menu, or may be user-defined, such as via numerical input from a user, such as via a keyboard. In an embodiment, computing device 114 may receive user input defining a desired or planned cut plane. For example, during tibial resection planning, computing device 114 may receive user input to define planned degrees of tibial slope. The heat map's color gradations may then represent the position of each point in the mesh along an axis normal to a planned cut plane. In another embodiment, the heat map's color gradations may represent the position of each point in the mesh along an axis normal to a trackable cut plane. Positional referencing to a trackable cut plane is discussed further below. The user may switch back-and-forth between anatomic and/or reference planes and/or anatomic and/or other reference axes to be used as the reference plane or axis for the color gradations of the heat map by providing input to computing device 114 via a selection from a list or drop-down or by user input via a keyboard or mouse. Computing device 114 may update the heat maps accordingly, in response to user input.

In an embodiment, heat maps 502 and 504 may be displayed relative to anatomic landmarks, such as the tibia center 310 and/or AP axis 406. For heat maps generated from meshes generated for one or more regions of femur 312, the heat maps may be displayed relative to anatomic landmarks such as the femur center 322 (see FIG. 3B for approximate location) and/or Whiteside's line.

As discussed previously regarding the positioning of displays of the one or more pluralities of anatomic points in relation to FIG. 4, heat maps 502 and 504 may be arranged to align with the positioning of the patient. Alternatively, heat maps 502 and 504 may not be arranged to align with the positioning of the patient. Further, in another embodiment, heat maps 502 and 504 may be arranged such that heat map 502 is positioned above heat map 504, or vice versa. The skilled person will readily appreciate that any number of heat maps, such as 2, 3 or 4 heat maps may be arranged in any way to display the same number of meshes. For example, embodiments involving femoral resection may comprise multiple heat maps, such as for each of the distal portion of the medial femoral condyle, the distal portion of the lateral femoral condyle, the posterior portion of the medial femoral condyle and the posterior portion of the lateral femoral condyle.

In an embodiment, computing device 114 may be configured to evaluate a plurality of points to determine the validity of each anatomic point. For example, a potential issue that arises when tracing the surface of anatomic structure is lift-off, which occurs when the probe tip loses contact with, or is lifted off the surface of the anatomic structure during tracing. Such lift-off points may be detected by computing device 114 based on surface smoothness or a threshold change in a point's position compared to one or more neighboring or surrounding points, particularly in the direction normal to the surface of the anatomic structure. The skilled person will understand that any other technique known in the art for detecting lift-off may be used. In an embodiment, anatomic points identified as lift-off points may be excluded, or filtered from the mesh generated for a region. The resulting heat map, such as heat map 552 generated for a lateral tibial plateau, is shown in tab 550 of UI 400 in FIG. 5B, illustrates a heat map with lift-off points excluded. Where lift-off has occurred, the anatomic distal point may not be accurately captured. By providing a visual indication that some anatomic points are lift-off points and therefore not valid, the user may be alerted that the registration of the surface may be incomplete or inaccurate. The skilled person will understand that other error detection methods may be used to evaluate the validity of anatomic points instead of or in addition to methods to detect lift-off, and these other invalid points may be excluded instead of or in addition to points determined to be invalid due to lift-off. As shown in FIG. 5B, white space 554 indicates a region where anatomic points were excluded due to invalidity, such as due to lift-off. The skilled person will appreciate that anatomic points may simply not have been collected for a portion of the region, as opposed to having been excluded due to lift-off. In either case, subsequent calculations using the mesh generated may not be accurate. In an embodiment, when anatomic points are determined to be invalid, UI 400 may prompt the user to collect additional anatomic points by retracing the region.

In accordance with an embodiment, such as the embodiment shown in FIG. 5A, computing device 114 may perform calculations using the mesh generated for each region to determine a dynamic distal point or a dynamic posterior point for each region. In an embodiment, dynamic distal points are determined relative to a cut plane (instead of a mechanical axis of the anatomic structure such as for the anatomic distal points discussed previously), such as while the user performs resection planning. As discussed above, the cut plane may be indicated by probe base 204 of trackable probe 200, which may be coupled to cutting guide 208 or cutting guide 208 and paddle guide 212, as discussed previously in relation to FIG. 2A-D. Alternatively or in addition, the cut plane may be indicated by a trackable cutting guide, or any other suitable trackable surgical instrument comprising a feature that may reasonably represent a cut plane. Therefore, a trackable probe comprising a probe base, such as probe base 204, a trackable cutting guide, or another suitable surgical instrument comprising a feature that may reasonably represent a cut plane may provide (e.g. indicate) a trackable cut plane. It is understood that the cut plane is determined as a plane extending from the tracked surgical instrument such as a plane extending from a probe base. The pose of the tracked surgical instrument may be determined from the pose of the tracker coupled to the tracked surgical instrument (or natural trackable features of the tracked surgical instrument) and the geometry of the tracked surgical instrument. The cut plane is thus indicated or provided by the tracked surgical instrument in that the cut plane is related to the pose of the tracked surgical instrument and its geometry.

In an embodiment, the dynamic tibial distal points (i.e. the dynamic lateral tibial distal point and the dynamic medial tibial distal point) represent the points in the respective meshes that lie nearest to the cut plane (i.e. measured in a direction normal to the cut plane). In an embodiment, the dynamic tibial distal points may be used to calculate the resection depth of the proximal tibia based on the pose of a surgical instrument providing a trackable cut plane, such as trackable probe 200 comprising probe base 204 and the pose of a tracker coupled to the patient's tibia. For example, a tibial dynamic distal point may be determined for each of the medial and lateral plateaus by selecting a point with the shortest distance to the cut plane from each of their respective meshes.

Alternatively or in addition, the dynamic femoral distal points (i.e. the dynamic lateral femoral distal point and the dynamic medial femoral distal point) represent the points in the respective meshes that lie farthest from the cut plane (i.e. measured in a direction normal to the cut plane). In an embodiment, the dynamic femoral distal points may be used to calculate the resection depth of the distal femur based on the pose of a surgical instrument providing a trackable cut plane, such as trackable probe 200 comprising probe base 204 and the pose of a tracker coupled to the patient's femur. For example, a femoral dynamic distal point may be determined for each of the medial and lateral femoral condyles by selecting a point with the largest distance to the cut plane from each of their respective meshes.

Similarly, the dynamic femoral posterior points (i.e. the dynamic lateral femoral posterior point and the dynamic medial femoral posterior point) represent the points in the respective meshes that lie farthest from the cut plane. In an embodiment, a femoral dynamic posterior point may be determined for each of the medial and lateral femoral condyles by selecting a point with the largest distance to the cut plane from each of the respective meshes for the posterior portions of the medial and lateral femoral condyles.

In an embodiment, computing device 114 performs, continuously and in real-time, the necessary calculations to determine the pose of trackable probe 200 comprising probe base 204 held by the user relative to a tracker coupled to the patient's anatomic structure. As the user maneuvers trackable probe 200, such as while performing resection planning, the dynamic distal points (and the corresponding resection depth) may be updated in real time. Computing device 114 may display the position of the dynamic distal points to the user via a UI, such as UI 400. For example, in accordance with the embodiment discussed in relation to FIG. 5A, a dynamic medial tibial distal point 508 may be determined by selecting the point nearest to the cut plane from the mesh generated for the medial tibial plateau (i.e. a first region). A dynamic lateral tibial distal point 510 may also be determined, such as by selecting the point nearest to the cut plane from the mesh generated for the lateral tibial plateau (i.e. a second region). The resulting dynamic medial tibial distal point 508 and the dynamic lateral tibial distal point 510 may be displayed via tab 500 of UI 400. In FIG. 5A, the dynamic tibial distal points 508 and 510 are indicated by square-shaped graphical indicators in display box 512, but could be any other appropriate shape, as discussed previously regarding graphical indicators.

The position of a dynamic distal point may be used to verify whether the mesh is an accurate representation of the underlying anatomic structure or surface (i.e. the mesh may not adequately represent the surface of the anatomic structure in that region or provide adequate coverage of the region). For example, a dynamic distal point that is positioned on the edge of a heat map may indicate that the plurality of anatomic points collected by the user may not sufficiently cover the anatomic surface. In other words, the most distal point of the surface may not have been captured by the user during surface registration (e.g. tracing or painting). Computing device 114 may perform the necessary calculations to detect that a dynamic distal point lies on an edge and may alert a user via UI 400. For example, UI 400 may alert the user via a visual indication (i.e. presentation of a visual message; a color change of a graphical indicator, for example, from green to red, etc.) or an auditory indication (i.e. a chime, an audible message, etc). In an embodiment, UI 400 may prompt the user to collect additional anatomic points by retracing the region (i.e. using probe tip 202, as previously discussed). Alternatively, UI 400 may not provide an alert. However, the presentation of the planar profiles with dynamic distal points overlaid enables the user to visually inspect the planar profile and assess whether the mesh is an accurate representation of the underlying anatomic structure or surface. An anatomic distal point may also be used as an indication of whether the mesh is an accurate representation of the underlying anatomic structure or surface in a manner similar to that of a dynamic distal point.

One or more profiles may be generated from a mesh to represent the surface contour through a slice of the anatomic structure. The profiles may be displayed to the user. Presenting profiles of the femoral condyles and/or tibial plateaus to the user, such as during resection planning, may allow identification of areas of wear that can be compensated for to achieve more accurate resection depths. Further, profiles may assist practitioners of the kinematic alignment philosophy to achieve native joint alignment. For example, profiles generated for posterior portions of the femoral condyles may assist users to achieve native rotation of the installed implant by identifying and compensating for asymmetrical wear.

Profiles may be planar or non-planar.

In an embodiment, one or more planar profiles may comprise anatomic and/or interpolated points lying within a defined distance threshold (e.g., within 0.5 mm) of a slice through the mesh; wherein the position and orientation of the slice is defined relative to one or more anatomic landmarks (e.g. an anatomic distal point, a dynamic distal point, etc.), anatomic reference planes (e.g. a coronal plane, a sagittal plane and/or a transverse plane), anatomic axes (e.g. a mechanical axis of the anatomic structure, an AP axis, Whiteside's line, an ML axis, etc.), and/or another reference axis or plane (e.g. a trackable cut plane, a plane defined by features of the mesh, or a user-defined plane). The distance threshold may be 0.5 mm, 0.75 mm, 1 mm, or any other suitable distance based on the distribution of points of the mesh. The one or more planar profiles may comprise an anatomic landmark, such as an anatomic distal point or a dynamic distal point. In other words, one or more planar profiles may be defined to intersect an anatomic landmark, such as an anatomic or dynamic distal point. Alternatively, a planar profile may not intersect an anatomic landmark, but instead may be further defined by an offset from one or more anatomic reference planes and/or anatomic axes. The offset may be pre-defined in the computing device 114 or may be provided via user input, such as via drop-down menu, manual entry using a keyboard, etc., in a UI.

For example, a planar profile may be defined by a slice through the mesh that is parallel to an AP axis. The slice may further be parallel to a mechanical axis of the anatomic structure and may intersect a dynamic distal point. The skilled person will appreciate that the one or more planar profiles are not restricted to a parallel relationship with one or more anatomic reference planes and/or anatomic axes but may instead may have any other angular relationship to one or more anatomic axes and/or anatomic or other reference planes and/or axes. For example, a planar profile may be defined by a slice through a mesh at an angle of 30 degrees, 60 degrees, or any other suitable angle to any reference plane and/or axis.

In an embodiment, a planar profile may not be defined by a slice relative to an anatomic landmark, an anatomic reference plane or an anatomic axis. Instead, the position and/or orientation of a slice may be defined by one or more features of the mesh. For example, in an embodiment, a planar profile may be defined by a slice that intersects a most anterior point and a most posterior point of the mesh. Alternatively, a planar profile may be defined by a slice that intersects a most medial point and a most lateral point of a mesh.

Any suitable number of planar profiles may be generated for one or more meshes. For example, in an embodiment, a first planar profile may be generated from a first mesh of the medial tibial plateau 304 and a second planar profile may be generated from a second mesh of the lateral tibial plateau 306. Both the first and second planar profiles may be defined by slices through their respective meshes that are parallel to an AP axis and the mechanical axis of the tibia. The first and second planar profiles may be further defined to intersect the dynamic medial tibial distal point and the dynamic lateral tibial distal points, respectively.

In another embodiment, two or more planar profiles may be generated from the same mesh. In an embodiment, first and second planar profiles may be generated from a mesh of the medial tibial plateau 304 (i.e. a first mesh). A first planar profile may be defined by a slice through the first mesh that is parallel to both the AP axis and the mechanical axis of the tibia. A second planar profile may be defined by a slice through the same mesh and may be parallel to both an ML axis and the mechanical axis of the tibia. The first and second planar profiles may be further defined to intersect a dynamic medial tibial distal point. Similar planar profiles may be generated from a second mesh of the lateral tibial plateau. For example, third and fourth planar profiles may be generated that are defined by slices that are parallel to the AP axis and the ML axis, respectively, and intersect a dynamic lateral tibial distal point.

The skilled person will readily appreciate that there can be any number of planar profiles generated for a given mesh and that similar planar profiles may be generated for each of two or more meshes generated to represent the surface contour of an anatomic structure of a patient.

One or more planar profiles may be displayed via a UI, such as UI 400. In the embodiment shown in FIG. 5A, the anatomic structure is a tibia. Tab 500 of UI 400 may assist a user in tibial resection planning. A first planar profile 518 may be generated from a mesh of the medial tibial plateau 304 (i.e. a first mesh generated for a first region) and a second planar profile 516 may be generated from a mesh of the lateral tibial plateau 306 (i.e. a second mesh generated for a second region), such as for proximal tibial resection planning during a TKA. First planar profile 518 may intersect the dynamic medial tibial distal point 508 and may comprise anatomic and/or interpolated points lying along slice 522 (i.e. within a distance threshold, such as within 0.5 mm), wherein slice 522 lies parallel to both an AP axis and the mechanical axis of the tibia (i.e. in FIG. 5A, slice 522 is perpendicular to the page and so is shown as a line). Second planar profile 516 may intersect the dynamic lateral tibial distal point 510 and may further comprise anatomic and/or interpolated points lying along slice 520 (i.e. within a distance threshold, such as within 0.5 mm), wherein slice 520 lies parallel to both an AP axis and the mechanical axis of the tibia (i.e. in FIG. 5A, slice 520 is perpendicular to the page and so is shown as a line). In an embodiment, the one or more planar profiles 518 and 516 may be displayed simultaneously via a UI, such as UI 400. Dynamic distal points 508 and 510 are indicated as square-shaped graphical indicators and are overlaid on both slices 522 and 520, respectively, as well as planar profiles 518 and 516, respectively. The graphical indicators may be any other shape, as previously discussed.

In an embodiment, planar profiles 518 and 516 may be displayed relative to a trackable cut plane. As previously discussed, a trackable cut plane may be indicated by, for example, probe base 204 or other surgical instrument whose pose is used to determine the trackable cut plane. For example, planar profiles 516 and 518 display the position of each anatomic and/or interpolated point included in the planar profile relative to probe base 204 (i.e. the distance between each point and probe base 204 in a direction normal to probe base 204). The pose of probe base 204 relative to planar profiles 518 and 516 is graphically represented in two dimensions as trackable cut plane 506 in FIG. 5A. In an embodiment, the planar profiles may intersect a dynamic distal point (also determined relative to trackable cut plane 506). In another embodiment, the planar profiles may intersect any appropriate anatomic landmark or reference point.

In an embodiment, the position of a dynamic distal point on a planar profile may be used to verify whether the mesh is an accurate representation of the underlying anatomic structure or surface (i.e. the mesh may not adequately represent the surface of the anatomic structure in that region or provide adequate coverage of the region), as discussed previously regarding the positioning of a dynamic distal point on the edge of a mesh. For example, a dynamic distal point positioned on the edge of a planar profile may indicate that the plurality of anatomic points collected by the user may not sufficiently cover the anatomic surface. In other words, the surface may not have been properly captured by the user during surface registration. Computing device 114 may perform the necessary calculations to detect that a dynamic distal point lies on an edge of a planar profile and may alert a user via UI 400. For example, UI 400 may alert the user via a visual indication (e.g. presentation of a visual message; a color change of a graphical indicator, for example, from green to red, etc.) or an auditory indication (e.g. a chime, an audible message, etc). In an embodiment, UI 400 may prompt the user to collect additional anatomic points by retracing the region (i.e. using probe tip 202, as previously discussed). Alternatively, UI 400 may not provide an alert. However, the presentation of the planar profiles with dynamic distal points overlaid enables the user to visually inspect the planar profile and assess whether the mesh is an accurate representation of the underlying anatomic structure or surface.

Referring to FIG. 5B, a planar profile may also provide a visual indication that erroneous data has been excluded or filtered, such as data identified as lift-off data. As discussed above, the skilled person will appreciate that anatomic and/or interpolated may be excluded for other reasons, including but not limited to that the anatomic points were not collected for a portion of the region. In the embodiment shown in FIG. 5B, profile 556 illustrates a planar profile with a gap corresponding to white space 554. As previously discussed, white space 554 indicates a region where anatomic points were excluded from the mesh. The gap in profile 556 also shows that anatomic points have been excluded, such as due to lift-off or were not collected. By providing a visual indication that some anatomic and/or interpolated points have been excluded due to invalidity or represent a portion of a region where anatomic points were not collected, the user may be alerted that the resulting planar profile may be inaccurate or incomplete. In an embodiment, the gap due missing or excluded anatomic and/or interpolated points may be represented by a dashed line, a dotted line, or any other graphical representation connecting the points on either side of the gap (not shown). In an embodiment, when anatomic and/or interpolated points are determined to be invalid, or a planar profile is determined to be incomplete, UI 400 may prompt the user to collect additional anatomic points by retracing the region.

Referring once again to FIG. 5A, display box 514 may comprise graduated lines to indicate resection depth. Graduated lines 532 may be spaced evenly (shown) or unevenly (not shown). Even spacing may be set at an any increment, such as 3 mm, 4 mm, 5 mm, or any other appropriate increment. Graduated lines 532 are parallel with trackable cut plane 506 to facilitate graphical monitoring of the change in planned resection depth as the user repositions probe base 204 (or other surgical instrument providing a trackable cut plane) while performing resection planning.

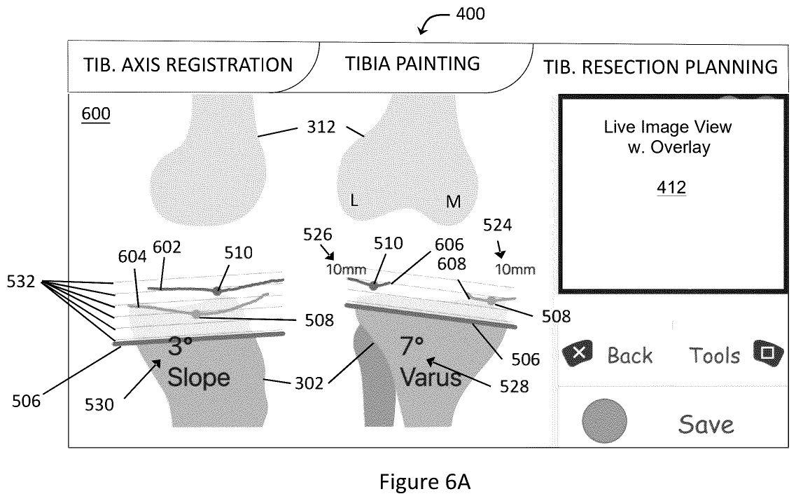

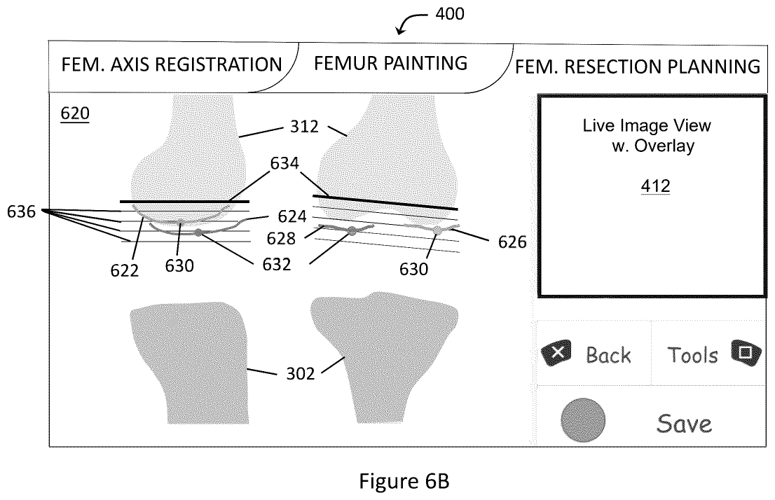

In an embodiment, the UI updates in real-time as the user maneuvers and/or repositions probe base 204 (or other surgical instrument providing a trackable cut plane), such as while performing resection planning. In an embodiment, as previously discussed, the planar profiles may be generated, in part, based on the location of dynamic distal points. Because the locations of the dynamic distal points are determined relative to trackable cut plane 506, both the dynamic distal points and the planar profiles may change as probe base 204 (or other surgical instrument providing a trackable cut plane) is repositioned. As the user repositions the trackable cut plane, computing device 114 continuously and in real-time performs the necessary computations to determine the locations of the dynamic distal points. As the trackable cut plane is moved, the dynamic distal points and the planar profiles that intersect the dynamic distal points may move in response. In an embodiment, such as shown in FIG. 5A, trackable cut plane 506 may be displayed as a stationary 2D line on tab 500 of UI 400, while the positions of the dynamic distal points and the planar profiles may be updated in real-time as probe base 204 is repositioned. To be clear, the actual cut plane, as indicated by probe base 204 (or other surgical instrument providing a pose for determining the trackable cut plane) in real space exists in 3 dimensions and does not remain stationary. In another embodiment, such as discussed below in relation to FIGS. 6A and 6B, the trackable cut plane may not be stationary and instead may be updated in real-time as the user repositions the trackable cut plane (e.g. probe base 204).

The display of at least two planar profiles simultaneously provides visual cues to the user to assist with resection planning that are not available with a single planar profile. For example, a user may refer to the relative position of the planar profiles while planning a resection to determine how to reposition the trackable cut plane (e.g. a trackable cutting guide 208 or other or probe base 204) to achieve target measurements, such as to achieve a desired surgical outcome (i.e. to accommodate an implant of a specific size and shape or to meet other surgical objectives). For example, a surgeon adhering to a kinematic alignment philosophy may align the profiles to match medial and lateral tibial resection depths in order to restore native anatomic alignment of the post-surgical joint.

In an embodiment, tab 500 of UI 400 is configured to display various measured parameters relevant to a user during a joint procedure, such as a TKA. For example, in the embodiment depicted in FIG. 5A, tab 500 is configured to display varus 528, slope 530, medial resection depth 526 and lateral resection depth 524, which are updated as probe base 204 (or other surgical instrument providing a trackable cut plane) is moved or repositioned. In the embodiment depicted in FIG. 5A and at the instant shown, medial and lateral resection depths 524 and 526, respectively, are both 9 mm. A user may refer to the measured parameters while planning a resection to accommodate an implant of a specific size and shape or to meet other surgical objectives.

In the embodiments depicted in FIGS. 5A and 5B, heat maps and planar profiles are displayed on the same tab, tab 500 and tab 550, respectively. However, the skilled person will appreciate that heat maps and planar profiles may be displayed on separate tabs in some embodiments. Further, the skilled person will appreciate that in some embodiments, only heat maps will be displayed on a particular tab and in other embodiments, only planar profiles will be displayed on a particular tab. Non-planar profiles may be displayed alternatively or in addition to planar profiles. Non-planar profiles are further discussed below.

The embodiments shown in FIGS. 5A and 5B relate to tibial resection planning, however, the skilled person will appreciate that the discussion herein relating to the generation of planar profiles involved in tibial resection planning also applies to femoral resection planning. For example, in an embodiment involving femoral resection planning, one or more planar profiles may be generated from meshes of one or more regions of the patient's femur (i.e. regions of the femur as discussed in relation to FIG. 3B). For example, a first planar profile may be generated from a first mesh of the distal portion of a medial femoral condyle 314 and a second planar profile may be generated from a second mesh of the distal portion of a lateral femoral condyle 318, Both the first and second planar profiles may be defined by slices through their respective meshes that are parallel to Whiteside's line and the mechanical axis of the femur. The first and second planar profiles may be further defined to intersect the dynamic medial femoral distal point and the dynamic lateral femoral distal point, respectively. Alternatively or in addition, a third planar profile may be generated from a third mesh of the posterior portion of the medial femoral condyle 316 and a fourth planar profile may be generated from a fourth mesh of the posterior portion of the lateral femoral condyle 320. Both the third and fourth planar profiles may be defined by slices through their respective meshes that are parallel to an ML axis and the mechanical axis of the femur. The third and fourth planar profiles may be further defined to intersect the dynamic medial femoral posterior point and the dynamic lateral femoral posterior point, respectively. The skilled person will readily appreciate that there can be any number of planar profiles generated for a given mesh and that similar planar profiles may be generated for each of two or more meshes generated to represent the surface contour of a femur of a patient. In an embodiment, two or more planar profiles may be displayed simultaneously via a UI, such as UI 400. In an embodiment, the planar profiles generated from meshes of one or more regions of the patient's femur may display the position of each anatomic and/or interpolated points included in the planar profile relative to a trackable cut plane (i.e. the distance between each point and the trackable cut plane). One or more planar profiles may each intersect a dynamic distal point (also determined relative to the trackable cut plane) or any other appropriate anatomic landmark or reference point.

FIG. 6A presents an embodiment, in which multiple planar profiles are displayed, such as for tibial resection planning. As shown, planar profiles 604 (i.e. a first planar profile) and 608 (i.e. a second planar profile) may both intersect and therefore comprise dynamic medial tibial distal point 508. Planar profile 604 may be further defined by a slice through the mesh that is parallel to both an AP axis and the mechanical axis of the tibia. Planar profile 608 may be further defined by a slice through the mesh that is parallel to both the ML axis of the tibia and the mechanical axis of the tibia. Similarly, planar profiles 602 (i.e. a third planar profile) and 606 (i.e. a fourth planar profile) may both intersect and therefore comprise dynamic lateral tibial distal point 510. Planar profile 602 may be further defined by a slice that is parallel to both an AP axis and the mechanical axis of the tibia. Planar profile 606 may be further defined as a slice that is parallel to both the ML axis of the tibia and the mechanical axis of the tibia. In an embodiment, two or more of planar profiles 602, 604, 606 and 608 may be displayed simultaneously via a UI, such as UI 400, to assist the user with tibial resection planning. The skilled person will appreciate that any appropriate number of planar profiles may be generated from each of one or more meshes and some or all may be displayed via a UI.

Planar profiles 602, 604, 606 and 608 may display the position of each anatomic and/or interpolated point relative to the mechanical axis of the tibia (i.e. in a direction parallel to the mechanical axis of the tibia). This is in contrast to the display for planar profiles 516 and 518 in FIG. 5A, which displays the position of each anatomic and/or interpolated point relative to trackable cut plane 506 (i.e. distance of each point to the cut plane).

FIG. 6A may include a similar graphical indicator for trackable cut plane 506 as that provided in FIG. 5A. However, in FIG. 6A, unlike in FIG. 5A, trackable cut plane 506 is dynamic. As a trackable cut plane such as probe base 204 (or other surgical instrument providing a trackable cut plane) is repositioned by the user, the graphical representation of the trackable cut plane, trackable cut plane 506, is adjusted to reflect the updated position and orientation of the trackable cut plane. As trackable cut plane 506 is adjusted, the position of the dynamic distal point may be adjusted (i.e. dynamic distal points are determined relative to the trackable cut plane), which may, in turn, alter the position of the planar profile (i.e. where the planar profile is defined to intersect a dynamic distal point). Graduated lines 532, as previously discussed, may be parallel to cut plane 506 and may be evenly or unevenly spaced. Evenly spaced graduation lines may be spaced at any increment, such as 3 mm, 4 mm, 5 mm or any other suitable increment. Further, graduated lines 532 may move relative to the motion of the trackable cut plane, such as probe base 204 (or other surgical instrument providing a trackable cut plane) such that they remain parallel to trackable cut plane 506.

In an embodiment, such as the embodiment of FIG. 6B, the anatomic structure may be a femur. One or more meshes may be generated for one or more regions of the patient's femur 312 (i.e. a mesh for each region) from one or more pluralities of anatomic points and/or interpolated points. For example, a first mesh may be generated for the distal portion of the medial femoral condyle 314 and a second mesh may be generated for the distal portion of the lateral femoral condyle 318. One or more planar profiles may be generated from each of the first and second meshes. For example, a first planar profile and a second planar profile may be generated from the first mesh and a third planar profile and a fourth planar profile may be generated from the second mesh. The one or more planar profiles may be displayed via a UI, such as via UI 400.

FIG. 6B presents an embodiment in which multiple planar profiles are displayed, in accordance with an embodiment involving femoral resection planning. As shown, planar profiles 622 (i.e. a first planar profile) and 626 (i.e. a second planar profile) have been generated from a mesh (i.e. a first mesh) generated for the distal portion of the medial femoral condyle. Planar profiles 622 and 626 may both intersect and therefore comprise dynamic medial femoral distal point 630. Planar profile 622 may be further defined by a slice that is parallel to both Whiteside's line and the mechanical axis of the femur. Planar profile 626 may be further defined by a slice that is parallel to both the ML axis and the mechanical axis of the femur. Similarly, planar profiles 624 (i.e. a third planar profile) and 628 (i.e. a fourth planar profile) have been generated from a mesh (i.e. a second mesh) generated for the distal portion of the lateral femoral condyle. Planar profiles 624 and 628 may both intersect and therefore comprise dynamic lateral femoral distal point 632. Planar profile 624 may be further defined by a slice that is parallel to both Whiteside's line and the mechanical axis of the femur. Planar profile 628 may be further defined by a slice that is parallel to both the ML axis and the mechanical axis of the femur. In an embodiment, two or more of planar profiles 622, 624, 626 and 628 may be displayed simultaneously via a UI, such as UI 400 to assist the user with femoral resection planning. The skilled person will appreciate that any appropriate number of planar profiles may be generated from each of one or more meshes and some or all may be displayed via a UI.

Similar to FIG. 6A, planar profiles 622, 624, 626 and 628 may display the position of each anatomic and/or interpolated point relative to the mechanical axis of the femur (i.e. in a direction parallel to the mechanical axis of the femur). FIG. 6B may include a similar graphical indicator for trackable cut plane 634 as that provided in FIG. 5A for trackable cut plane 506. Also similar to FIG. 6A, trackable cut plane 634 is dynamic. As a trackable cut plane such as probe base 204 (or other surgical instrument providing a trackable cut plane) is repositioned by the user, trackable cut plane 634 is adjusted in real-time to reflect the updated position of the trackable cut plane. As the position of probe base 204 (or other surgical instrument providing a trackable cut plane) is adjusted, the position of the dynamic distal point may be adjusted (i.e. dynamic distal points are determined relative to the cut plane), which may, in turn, alter the position of the planar profile (i.e. the planar profile defined to intersect a dynamic distal point). Graduated lines 636, which are similar to graduated lines 532 (previously discussed), may be evenly or unevenly spaced. Evenly spaced graduation lines may be spaced at any increment, such as 3 mm, 4 mm, 5 mm, or any other suitable increment. Further, graduated lines 636 may move relative to the motion of trackable cut plane 634 such that they remain parallel to trackable cut plane 634.

In an embodiment, one or more non-planar profiles may be derived from one or more meshes. For example, a non-planar profile may track a ridge or a groove across an anatomic surface instead of being defined by a slice that is parallel with an anatomic axis or plane. The non-planar profiles may be displayed on a UI (not shown). Various features and elements discussed previously related to displaying planar profiles applies to displaying non-planar profiles, such as the display of graduation lines, anatomic and/or dynamic distal points, resection depths, etc.

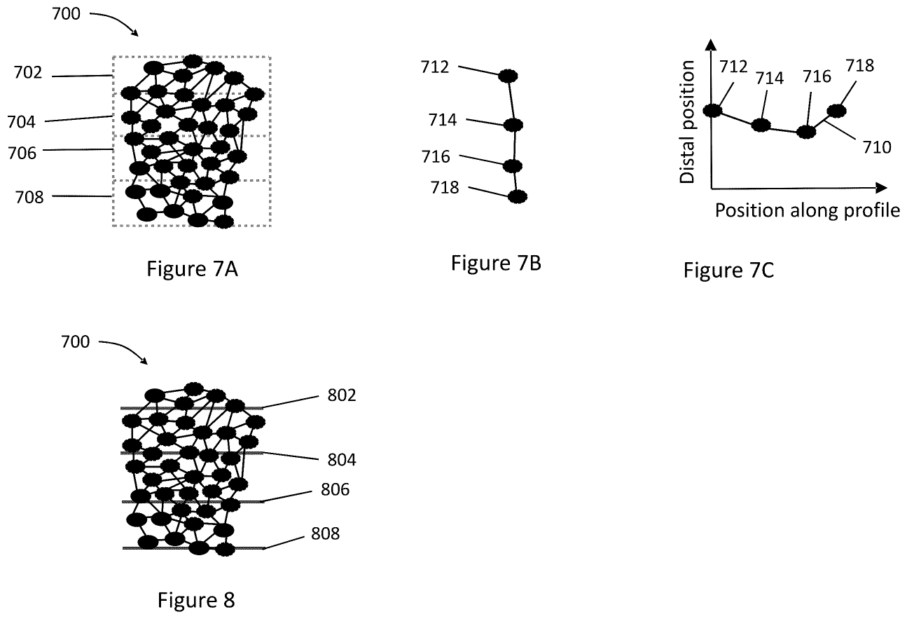

In an embodiment, computing device 114 may generate a non-planar profile by first determining the anatomic distal point of each of two or more areas of a mesh and then assembling the anatomic distal points into a non-planar profile. In the embodiment shown in FIG. 7A, for example, mesh 700 is segregated into four approximately equally sized areas 702, 704, 706 and 708. In each of areas 702, 704, 706, and 708 an anatomic distal point is identified. As previously discussed, an anatomic distal point is identified as the most distal point of the mesh relative to the mechanical axis. Computing device 114 may determine anatomic distal points 712, 714, 716 and 718, one from each of areas 702, 704, 706 and 708, respectively, as illustrated in FIG. 7B. Computing device 114 may then generate non-planar profile 710 comprising anatomic distal points 712, 714, 716 and 718 shown plotted in FIG. 7C as distal position relative to the mechanical axis of the anatomic structure versus position along the profile. Non-planar profile 710 may then be displayed to the user via a UI (not shown). The non-planar profiles may be displayed in two dimensions or three dimensions. The skilled person will readily appreciate that a non-planar profile may be generated comprising anatomic distal points for each of any number of areas of a mesh. For example, a mesh may comprise five areas, six areas, ten areas, or any other suitable number of areas. Further, the areas may have any shape, such as triangular, square, rectangular (as shown in the FIG. 7A), or any other appropriate shape, and that the two or more areas may be the same size, or may have different sizes. The areas may be configured in any pattern, with areas arranged side-by-side, top-to-bottom, or in any other pattern. The number of areas may be pre-defined in the computing device 114 or may be user-selectable in UI 400, such as via drop-down menu, or use-defined in UI 400, such as via manual entry using a keyboard, etc. The skilled person will also appreciate that a non-planar profile may be generated comprising dynamic distal points, one for each of at least two areas of a mesh. As previously discussed, a dynamic distal point is identified as the most distal point of the mesh relative to a trackable cut plane.

For clarity, in an embodiment, one or more non-planar profiles may be generated wherein each non-planar profile may comprise an anatomic distal point or a dynamic distal point for every area of a mesh or for two or more, but not all areas of a mesh.

In another embodiment, computing device 114 may generate a non-planar profile by first determining an anatomic distal point for each of two or more planar profiles generated from a mesh. For example, two or more planar profiles may be defined by slices that are parallel to an ML axis or an AP axis and to each other. The slices may further be parallel to a mechanical axis of the anatomic structure. As shown in FIG. 8, in accordance with an embodiment, four planar profiles 802, 804, 806 and 808 have been generated from mesh 700. Planar profiles 802, 804, 806, and 808 are parallel to an ML axis and are shown approximately equally spaced. However, the skilled person will appreciate that the planar profiles may have variable spacing. Computing device 114 may determine an anatomic distal point for each planar profile based on identifying an anatomic point and/or an interpolated point lying within a pre-defined distance threshold of the planar profile. For example, the distance threshold may be defined to be 0.5 mm, 0.75 mm, 1 mm, or any other appropriate distance threshold, which may depend on the spacing of the planar profiles, the complexity of the region of the anatomic structure, or any other factor. Computing device 114 may generate a non-planar profile comprising the anatomic distal points of each planar profile. For example, computing device 114 may generate a non-planar profile comprising four anatomic distal points, one for each of planar profiles 802, 804, 806 and 808 (non-planar profile not shown). The non-planar profile may then be displayed to the user via a UI, such as UI 400 (not shown). The non-planar profiles may be shown in two dimensions or three dimensions. The skilled person will readily appreciate that a non-planar profile may be generated comprising anatomic distal points for each of any number of planar profiles generated from a mesh. The number of planar profiles and their spacing may be pre-defined in the computing device 114 or may be user-selectable in UI 400, such as via drop-down menu, manual entry using a keyboard, etc. The skilled person will also appreciate that the planar profiles may be defined by slices that are parallel to any anatomic axis or reference plane, such as an AP axis. The skilled person will also appreciate that a non-planar profile may, alternatively or in addition, be generated comprising dynamic distal points as opposed to anatomic distal points (i.e. one for each of at least two planar profiles generated from a mesh).

In an embodiment, a non-planar profile may be generated for each of any number of meshes. For example, where the anatomic structure is a tibia, a first non-planar profile may be generated for a first mesh of the medial tibial plateau 304 and a second non-planar profile may be generated for a second mesh of the lateral tibial plateau 306.

UI 400 may have any number and combination of tabs, and each tab may present information for one or more anatomic structures, such as a tibia 302 and/or a femur 312. The one or more tabs may present one or more pluralities of points, one or more meshes in the form of heat maps, one or more planar profiles, and/or one or more non-planar profiles, alone or in any combination.

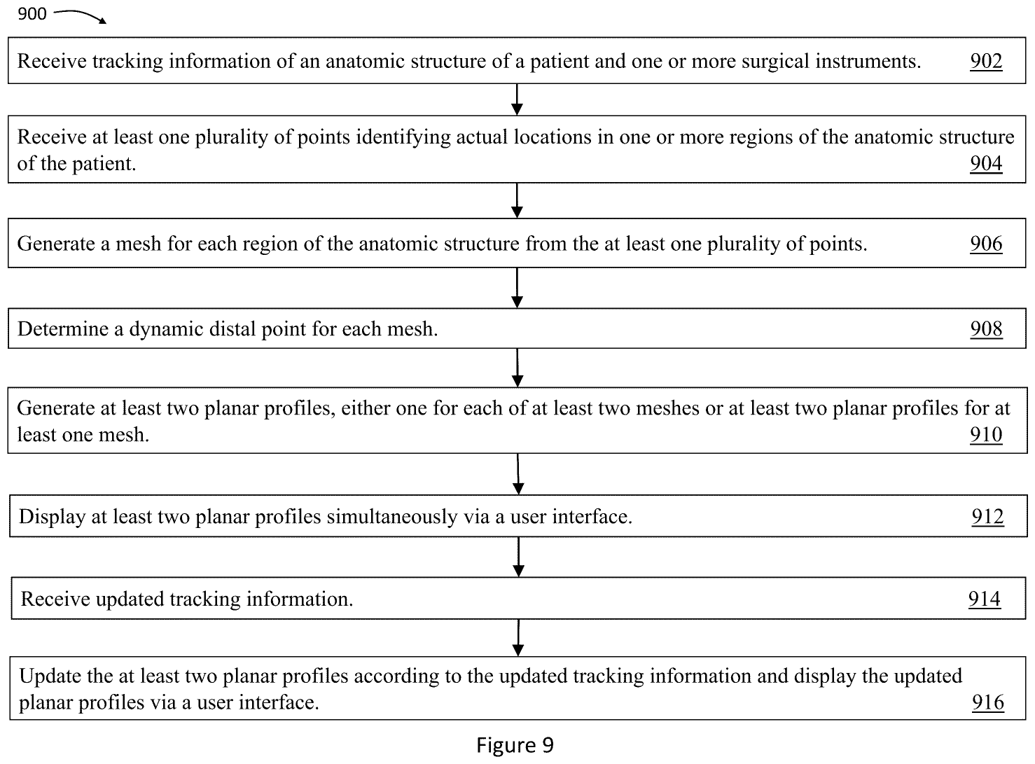

FIG. 9 is a flowchart of operations 900 of a method for intra-operative surgical planning in accordance with an embodiment. At step 902, computing device 114 receives tracking information of: an anatomic structure of a patient and one or more surgical instruments. As previously discussed, tracking information may comprise image data and/or pose data associated with the pose of one or more trackers and the objects to which they are attached. Trackers may be coupled to an anatomic structure of a patient and one or more surgical instruments. The one or more surgical instruments may comprise a probe tip, a probe base, a cutting guide, or any other surgical instrument providing a trackable cut plane.

At step 904, computing device 114 receives at least one plurality of anatomic points identifying actual locations in one or more regions of the anatomic structure of the patient. Receiving at least one plurality of anatomic points may comprise determining a location of probe tip 202 for each of the points in the at least one plurality of anatomic points. Alternatively, the at least one plurality of points may be received from a scanning system. Computing device 114 may optionally display the at least one plurality of anatomic points via a UI, such as UI 400.

At step 906, computing device 114 generates a mesh for each region of the anatomic structure of the patient. The one or more meshes may each comprise anatomic points and may further comprise interpolated points. Optionally, computing device 114 may evaluate each anatomic and interpolated point for validity, as previously discussed. Computing device 114 may exclude or filter points identified as lift-off points from the mesh and/or the plurality of anatomic points.

At step 908, a dynamic distal point may be determined for each mesh relative to a cut plane, wherein the cut plane may be indicated by probe base 204 of trackable probe 200 or any other surgical instrument providing a trackable cut plane (i.e. the pose data thereof is provided to determine the plane). One or more dynamic distal points (i.e. one for each region) may be determined based on tracking information of the one or more surgical tools and the tracking information of the anatomic structure of the patient. For example, a user may position a trackable cut plane such as probe base 204 (or a surgical instrument providing another trackable cut plane) on or adjacent to the anatomic structure of the patient while performing surgical planning. A dynamic distal point may be determined based on the pose of the surgical instrument providing a trackable cut plane (e.g. probe base 204) relative to the pose of the anatomic structure of the patient. Alternatively or additionally, an anatomic distal point may be determined for each mesh. The skilled person will appreciate that in some cases, such as for a mesh of the posterior portions of the femoral condyles, dynamic or anatomic posterior points may be determined instead of anatomic or dynamic distal points.

At step 910, computing device 114 generates one of: at least two planar profiles from each of one or more meshes; and at least one planar profile from each of at least two meshes. In an embodiment, each planar profile may comprise anatomic and/or interpolated points defined by a slice through the mesh; wherein the position and orientation of the slice is defined relative to one or more anatomic landmarks, anatomic reference planes and/or anatomic axes, and/or another reference axis or plane related to the pose of a surgical instrument. For example, in an embodiment, one or more planar profiles may each comprise a dynamic distal point of the respective mesh and may be defined by slices that are parallel to a mechanical axis of an anatomic structure and an AP axis. The skilled person will appreciate that a planar profile may comprise other anatomic landmarks and may be defined by a slice that is parallel to other anatomic axes and/or reference planes. At step 912, the planar profiles and optionally, the dynamic distal points may be displayed simultaneously via a UI.

As the user reorients or repositions the surgical instrument providing the trackable cut plane such as probe base 204 while performing surgical planning, computing device 114 receives updated tracking information of the anatomic structure and the one or more surgical instruments, as indicated at step 914. At step 916, computing device 114 updates the planar profiles according to the updated tracking information and displays the updated planar profiles via a UI.

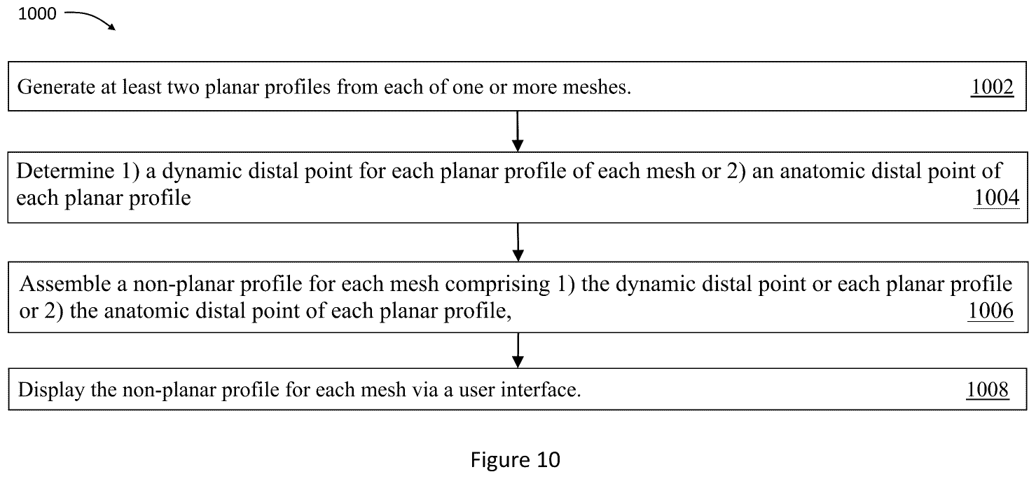

FIG. 10 is a flowchart of operations 1000 of a method for generating a non-planar profile for at least one mesh, in accordance with an embodiment. It is to be understood that the operations described in relation to FIG. 10 follow steps equivalent to that of steps 902 (i.e. receive tracking information of an anatomic structure of a patient and one or more surgical instruments), 904 (i.e. receive at least one plurality of points identifying actual locations in one or more regions of the anatomic structure of the patient) and 906 (i.e. generate a mesh for each region of the anatomic structure from the at least one plurality of points). At step 1002, computing device 114 generates at least two planar profiles for each of one or more meshes, wherein each planar profile is defined by a slice that is parallel to one or more anatomic axes or anatomic reference planes. The two or more planar profiles are parallel to each other and may be equally spaced or unequally spaced. At step 1004, computing device 114 determines a dynamic distal point of each planar profile of a mesh and/or an anatomic distal point of each mesh (e.g. either or both of such). At step 1006, computing device 114 assembles (i.e. generates) a non-planar profile for each mesh comprising one of: the dynamic distal points of each planar profile of the mesh and the anatomic distal points of each planar profile of the mesh. At step 1008, computing device 114 displays a non-planar profile for each mesh via a UI. The skilled person will appreciate that in some cases, such as for a mesh of the posterior portions of the femoral condyles, dynamic or anatomic posterior points may be determined instead of anatomic or dynamic distal points.

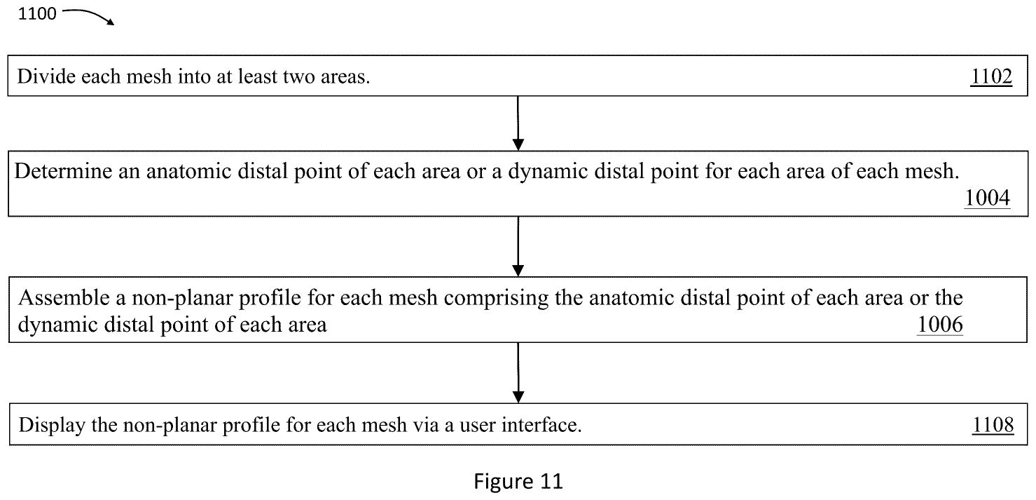

FIG. 11 is a flowchart of operations 1100 of a method for generating a non-planar profile for at least one mesh, in accordance with an embodiment. It is to be understood that the operations described in relation to FIG. 11 follow steps equivalent to that of steps 902 (i.e. receive tracking information of an anatomic structure of a patient and one or more surgical instruments), 904 (i.e. receive at least one plurality of points identifying actual locations in one or more regions of the anatomic structure of the patient) and 906 (i.e. generate a mesh for each region of the anatomic structure from the at least one plurality of points). At step 1102, computing device 114 divides each mesh into at least two areas. At step 1104, computing device 114 determines: an anatomic distal point for each area of each mesh and/or a dynamic distal point for each area of each mesh (e.g. either or both of such). At step 1106, computing device 114 assembles (i.e. generates) a non-planar profile for each mesh comprising one of: the anatomic distal points of each area and the dynamic distal points of each area. At step 1108, computing unit 114 displays the non-planar profile for each mesh via a UI. The skilled person will appreciate that in some cases, such as for a mesh of the posterior portions of the femoral condyles, dynamic or anatomic posterior points may be determined instead of anatomic or dynamic distal points.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise”, “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

1. A computer implemented method comprising:
receiving tracking information of an anatomic structure of a patient;
receiving tracking information of one or more surgical instruments;
receiving at least one plurality of anatomic points identifying actual locations in one or more regions of the anatomic structure of the patient;
generating a respective mesh of each of the one or more regions from the at least one plurality of anatomic points;
generating at least two planar profiles from one or more respective meshes, wherein each planar profile is defined to be a slice that is parallel to one or both of an anatomic axis or an anatomic reference plane;
displaying the at least two planar profiles simultaneously via a user interface;
receiving updated tracking information of the anatomic structure and the one or more surgical instruments;
updating the at least two planar profiles according to the updated tracking information; and
displaying the at least two updated planar profiles on a user interface.

2. The method of claim 1, comprising:
determining a dynamic distal point of each respective mesh relative to a trackable cut plane indicated by the one or more surgical instruments based on the tracking information of the anatomic structure and the tracking information of the one or more surgical instruments; and
wherein each planar profile from one respective mesh comprises the dynamic distal point of the one respective mesh.

3. The method of claim 1, wherein receiving the at least one plurality of anatomic points comprises determining a location of a probe tip of the one or more surgical instruments for each of the points of the at least one plurality of anatomic points.

4. The method of claim 1, wherein receiving the at least one plurality of anatomic points comprises receiving input from a scanner being manipulated to scan a region of the patient anatomy.

5. The method of claim 1, wherein the one or more surgical tools comprises at least two surgical tools; and wherein a first surgical tool comprises the probe tip and a second surgical tool indicates the trackable cut plane.

6. The method of claim 1, wherein the one or more surgical tools comprises one surgical tool; and wherein the one surgical tool comprises the probe tip and indicates the trackable cut plane.

7. The method of any of claim 1, wherein the trackable cut plane is indicated by a probe base.

8. The method of claim 1, wherein the anatomic structure is a tibia and the one or more regions comprises either or both of a medial tibial plateau or a lateral tibial plateau.

9. The method of claim 8, wherein one or more planar profiles are defined by a slice that is parallel to a mechanical axis of the tibia and is parallel to an anterior-posterior (AP) axis of the tibia.

10. The method of claim 8, wherein one or more planar profiles are defined by a slice that is parallel to a mechanical axis of the tibia and is parallel to a medial-lateral (ML) axis of the tibia.

11. The method of claim 1, wherein the anatomic structure is a femur, and the one or more regions are selected from a medial femoral condyle, a lateral femoral condyle, and a posterior femoral condyle.

12. The method of claim 11, wherein one or more planar profiles are defined by a slice that is parallel to a mechanical axis of the femur and is parallel to Whiteside's line.

13. The method of claim 11, wherein one or more planar profiles are defined by a slice that is parallel to a mechanical axis of the femur and is parallel to a ML axis of the femur.

14. The method of claim 1 further comprising:
determining a resection depth for each of the one or more regions based on one or more planar profiles; and
displaying at least one resection depth via a user interface.

15. The method of claim 1, wherein at least some of the respective meshes comprises interpolated points.

16. The method of claim 15 further comprising, for each of one or more respective meshes, displaying a first heat map for a first respective mesh via a user interface; wherein the first heat map displays a position of either or both of the anatomic points and the interpolated points of the first respective mesh relative to either a) the trackable cut plane or b) a mechanical axis of the anatomic structure.

17. A computer system comprising at least one processing unit and a memory coupled to at least one processing unit, a storage device storing instructions that, when executed by the at least one processing unit, cause the computer system to:
receive tracking information of an anatomic structure of a patient;
receive tracking information of one or more surgical instruments;
receive at least one plurality of anatomic points identifying actual locations in one or more regions of the anatomic structure of the patient;
generate a respective mesh of each of the one or more regions from the at least one plurality of anatomic points;
generate at least two planar profiles from one or more respective meshes, wherein each planar profile is defined to be a slice that is parallel to one or both of an anatomic axis or an anatomic reference plane;
display the at least two planar profiles simultaneously via a user interface;
receive updated tracking information of the anatomic structure and the one or more surgical tools;
update the at least two planar profiles according to the updated tracking information; and
display the at least two updated planar profiles on a user interface.

18. A computer implemented method comprising:
receiving tracking information of an anatomic structure of a patient;
receiving tracking information of one or more surgical instruments;
receiving at least one plurality of anatomic points identifying actual locations in one or more regions of the anatomic structure of the patient;
generating a respective mesh for each of the one or more regions from the at least one plurality of anatomic points;
generating a non-planar profile for each respective mesh; and
displaying one or more non-planar profiles via the user interface.

19. The method of claim 18 wherein generating the non-planar profile for each respective mesh comprises:
generating at least two planar profiles from each respective mesh; wherein each planar profile is defined by a slice that is parallel to either or both of: an anatomic axis or an anatomic reference plane;
determining either: 1) respective dynamic distal points of each of the at least two planar profiles relative to a trackable cut plane indicated by the one or more surgical instruments based on the tracking information of the anatomic structure and the tracking information of the one or more surgical instruments; or 2) respective anatomic distal points of each of the at least two planar profiles; and
assembling the non-planar profile for each respective mesh comprising either: 1) the respective dynamic distal points; or 2) the respective anatomic distal points.

20. The method of claim 18 wherein generating the non-planar profile for one respective mesh comprises:
dividing the one respective mesh into at least two areas;
determining respective anatomic distal points of each area; or respective dynamic distal points of each area for the non-planar profile for the one respective mesh.
